(19) United States
(12) Patent Application Publication
(10) Pub. No.: US 2024/0415674 A1
(43) Pub. Date: Dec. 19, 2024

(54) IMPLANTABLE INFLATABLE DEVICE

(71) Applicants: Boston Scientific Scimed, Inc., Maple Grove, MN (US); Boston Scientific Medical Device Limited, Galway (IE)

(72) Inventors: Amit Kumar, Noida (IN); Jonathan J. Lund, Glencoe, MN (US); Umesh Kumar Badaya, Dausa (IN)

(21) Appl. No.: 18/733,329

(22) Filed: Jun. 4, 2024

Related U.S. Application Data
(60) Provisional application No. 63/507,873, filed on Jun. 13, 2023.

(51) Int. Cl. A61F 2/48; A61F 2/26; A61M 5/168

(52) U.S. Cl. CPC A61F 2/484; A61F 2/26; A61M 5/16818; A61F 2250/0003

(57) ABSTRACT

An implantable fluid operated inflatable device may include a fluid reservoir configured to hold fluid, an inflatable member, and fluidics architecture that provides for transfer of fluid between the fluid reservoir and the inflatable member. The fluid reservoir, the inflatable member, and the fluidics architecture may be included in a single unit, or a single housing, thus providing for a relatively compact implantable fluid operated inflatable device. The relatively simple, compact device provides for simplified insertion into the patient, and for simplified operation by the user.

### CROSS-REFERENCE TO RELATED APPLICATION

This application claims priority to U.S. Provisional Patent Application No. 63/507,873, filed on Jun. 13, 2023, entitled “IMPLANTABLE INFLATABLE DEVICE”, the disclosure of which is incorporated by reference herein in its entirety.

### TECHNICAL FIELD

This disclosure relates, in general, to bodily implants, and in particular to inflatable bodily implants.

### BACKGROUND

Implantable fluid operated devices often include one or more valves, positioned within fluid passageways of the implantable device, to direct and control a flow of fluid between different portions of the implantable device. The flow of fluid to and from one or more fluid filled components of the implantable device may be controlled to achieve inflation, deflation, pressurization, depressurization, activation, deactivation and the like of the one or more fluid filled components of the implantable device. An implantable fluid operated device including a pump and valve system and a fluid reservoir in a single implantable device may provide for a simplified, less invasive insertion of the implantable device, and/or simplified user operation of the implantable device.

### SUMMARY

In some aspects, the techniques described herein relate to an implantable fluid operated inflatable device, including a housing; a fluid reservoir received in the housing; an inflatable member received in the housing; fluidics components provided in the housing, the fluidics components providing for fluid communication between the fluid reservoir and the inflatable member; and a fluid flow control system provided on the housing and configured to provide for fluid flow from the fluid reservoir to the inflatable member in a first mode of operation in response to an input at a first fluid flow control device of the fluid flow control system, and for fluid flow from the inflatable member to the fluid reservoir in a second mode of operation in response to an input at a second fluid flow control device of the fluid flow control system.

In some implementations, the implantable fluid operated inflatable device is a penile prosthesis, including: a proximal tip at a first end portion of the housing; a distal tip at a second end portion of the housing; an inflatable portion corresponding to the inflatable member, an intermediate portion of the housing.

In some implementations, the first fluid flow control device includes a malleable bulb provided in the distal tip; and the second fluid flow control device includes a valve actuation device provided at the intermediate portion of the housing.

In some implementations, the fluidics components include: a supply tube connecting the fluid reservoir and a bulb positioned in the distal tip; a first valve positioned in the supply tube; a port connecting the bulb to the inflatable member; a second valve positioned at the port connecting the bulb to the inflatable member; a fluid passageway connecting the inflatable member and the fluid reservoir; and a third valve positioned in the fluid passageway connecting the inflatable member and the fluid reservoir.

In some implementations, the first fluid flow control device includes the bulb provided in the distal tip, and wherein, in response to a first manipulation of the bulb positioned in the distal tip: the first valve is closed prevent fluid in the bulb from flowing into the supply tube; the second valve is opened to allow fluid held in the bulb to flow from the bulb to the inflatable member to inflate the inflatable member; and the third valve is closed to prevent fluid from flowing from the inflatable member to the fluid reservoir.

In some implementations, in response to a second manipulation of the bulb positioned in the distal tip: the first valve is opened to allow fluid to flow from the supply tube into the bulb to refill the bulb; the second valve is closed to prevent flow of fluid from the bulb into the supply tube; and the third valve is closed to prevent fluid from flowing from the inflatable member to the fluid reservoir.

In some implementations, the first manipulation of the bulb is a compression of the bulb and the second manipulation is a release of the compression of the bulb.

In some implementations, the second fluid flow control device includes a valve actuation device provided at the intermediate portion of the housing, and wherein, in response to a manipulation of the valve actuation device: the third valve is opened to allow fluid to flow from the inflatable member to the fluid reservoir to deflate the inflatable member; the first valve is closed prevent fluid in the bulb from flowing into the supply tube; and the second valve is closed to prevent fluid held in the bulb from flowing to the inflatable member.

In some implementations, the valve actuation device includes: a button portion provided in a recess formed in an outer peripheral surface of the housing; and at least one arm extending radially inward, into the housing, from the button portion, so as to selectively contact the third valve.

In some implementations, in an unactuated state of the valve actuation device, the third valve is positioned so as to close the fluid passageway between the inflatable member and the fluid reservoir.

In some implementations, in an actuated state of the valve actuation device, the at least one arm is moved radially inward in response to a depression of the button portion; the third valve moves to open the fluid passageway between the inflatable member and the fluid reservoir in response to a force exerted on the third valve by the at least one arm; and fluid flows from the inflatable member to the fluid reservoir to deflate the inflatable member.

In some implementations, the third valve is oriented axially within the housing, wherein the at least one arm is configured to exert a radial force on the third valve that moves the third valve in an axial direction to open the fluid passageway between the inflatable member and the fluid reservoir.

In some implementations, the third valve is oriented radially within the housing, and wherein the at least one arm is configured to exert a radial force on the third valve that moves the third valve in a radial direction to open the fluid passageway between the inflatable member and the fluid reservoir.

In some implementations, the button portion includes one of: a ring-shaped button arranged in the recess, extending around the intermediate portion of the housing in which the recess is formed; an arcuate button arranged in the recess, extending partially around the intermediate portion of the housing in which the recess is formed; or a plurality of arcuate buttons arranged in the recess, spaced apart along the intermediate portion of the housing in which the recess is formed.

In some implementations, the inflatable member includes: an inner wall and an outer wall defining an inflation tube that extends longitudinally along the intermediate portion of the housing; a plurality of first openings formed between the inner wall and the outer wall, extending longitudinally along a length of the inflatable member; a plurality of second openings formed between the inner wall and the outer wall, alternately arranged with the plurality of first openings, extending longitudinally along the length of the inflatable member; and a central opening defined by the inner wall, extending longitudinally along the length of the inflatable member, wherein, in an inflated state of the inflatable member, the plurality of first openings and the plurality of second openings are configured to be filled with fluid so as to inflate the inflatable member.

In some aspects, the techniques described herein relate to an implantable fluid operated inflatable device, including a housing; a fluid reservoir received in the housing; an inflatable member received in the housing; fluidics components provided in the housing, the fluidics components providing for fluid communication between the fluid reservoir and the inflatable member; and a fluid flow control system provided on the housing and configured to provide for fluid flow from the fluid reservoir to the inflatable member in a first mode of operation in response to an input at a first fluid flow control device of the fluid flow control system, and for fluid flow from the inflatable member to the fluid reservoir in a second mode of operation in response to an input at a second fluid flow control device of the fluid flow control system.

In some implementations, the implantable fluid operated inflatable device is a penile prosthesis, including: a proximal tip at a first end portion of the housing; a distal tip at a second end portion of the housing; an inflatable portion corresponding to the inflatable member, an intermediate portion of the housing.

In some implementations, the first fluid flow control device includes a malleable bulb provided in the distal tip; and the second fluid flow control device includes a valve actuation device provided at the intermediate portion of the housing.

In some implementations, the fluidics components include: a supply tube connecting the fluid reservoir and a bulb positioned in the distal tip; a first valve positioned in the supply tube; a port connecting the bulb to the inflatable member; a second valve positioned at the port connecting the bulb to the inflatable member; a fluid passageway connecting the inflatable member and the fluid reservoir; and a third valve positioned in the fluid passageway connecting the inflatable member and the fluid reservoir.

In some implementations, the first fluid flow control device includes the bulb provided in the distal tip, and wherein, in response to a first manipulation of the bulb positioned in the distal tip: the first valve is closed prevent fluid in the bulb from flowing into the supply tube; the second valve is opened to allow fluid held in the bulb to flow from the bulb to the inflatable member to inflate the inflatable member; and the third valve is closed to prevent fluid from flowing from the inflatable member to the fluid reservoir.

In some implementations, in response to a second manipulation of the bulb positioned in the distal tip, the first valve is opened to allow fluid to flow from the supply tube into the bulb to refill the bulb; the second valve is closed to prevent flow of fluid from the bulb into the supply tube; and the third valve is closed to prevent fluid from flowing from the inflatable member to the fluid reservoir.

In some implementations, the first manipulation of the bulb is a compression of the bulb and the second manipulation is a release of the compression of the bulb.

In some implementations, the second fluid flow control device includes a valve actuation device provided at the intermediate portion of the housing, and wherein, in response to a manipulation of the valve actuation device: the third valve is opened to allow fluid to flow from the inflatable member to the fluid reservoir to deflate the inflatable member; the first valve is closed prevent fluid in the bulb from flowing into the supply tube; and the second valve is closed to prevent fluid held in the bulb from flowing to the inflatable member.

In some implementations, the valve actuation device includes: a button portion provided in a recess formed in an outer peripheral surface of the housing; and at least one arm extending radially inward, into the housing, from the button portion, so as to selectively contact the third valve.

In some implementations, in an unactuated state of the valve actuation device the third valve is positioned so as to close the fluid passageway between the inflatable member and the fluid reservoir.

In some implementations, in an actuated state of the valve actuation device, the at least one arm is moved radially inward in response to a depression of the button portion; the third valve moves to open the fluid passageway between the inflatable member and the fluid reservoir in response to a force exerted on the third valve by the at least one arm; and fluid flows from the inflatable member to the fluid reservoir to deflate the inflatable member.

In some implementations, the third valve is oriented axially within the housing, wherein the at least one arm is configured to exert a radial force on the third valve that moves the third valve in an axial direction to open the fluid passageway between the inflatable member and the fluid reservoir.

In some implementations, the third valve is oriented radially within the housing, and wherein the at least one arm is configured to exert a radial force on the third valve that moves the third valve in a radial direction to open the fluid passageway between the inflatable member and the fluid reservoir.

In some implementations, the button portion includes one of: a ring-shaped button arranged in the recess, extending around the intermediate portion of the housing in which the recess is formed; an arcuate button arranged in the recess, extending partially around the intermediate portion of the housing in which the recess is formed; or a plurality of arcuate buttons arranged in the recess, spaced apart along the intermediate portion of the housing in which the recess is formed.

In some implementations, the inflatable member includes an inner wall and an outer wall defining an inflation tube that extends longitudinally along the intermediate portion of the housing; a plurality of first openings formed between the inner wall and the outer wall, extending longitudinally along a length of the inflatable member; and a plurality of second openings formed between the inner wall and the outer wall, alternately arranged with the plurality of first openings, extending longitudinally along the length of the inflatable member, wherein, in an inflated state of the inflatable member, the plurality of first openings and the plurality of second openings are configured to be filled with fluid so as to inflate the inflatable member.

In some implementations, the inner wall defines a central opening extending longitudinally along a length of the inflatable member, wherein at least some of the fluidics components are received within a space defined by the central opening.

In some aspects, the techniques described herein relate to a fluid flow control system for an implantable fluid operated inflatable device, including a housing; a first fluid flow control device provided at a distal end portion of the housing, the first fluid flow control device including: a bulb provided in distal tip defining the distal end portion of the housing; a first valve positioned in a supply tube connecting the bulb with a fluid reservoir; and a second valve positioned in a port connecting the bulb and an inflatable member; a second fluid flow control device provided at an intermediate portion of the housing, the second fluid flow control device including: a valve actuation device configured to selectively actuate a third valve provided in a fluid passageway connecting the inflatable member and the fluid reservoir, the valve actuation device including: a button portion provided in a recess formed in an outer peripheral surface of the housing; and at least one arm extending radially inward, into the housing, from the button portion, so as to selectively contact the third valve.

In some implementations, in a first mode of operation of the implantable fluid operated inflatable device, in response to a compression of the bulb positioned in the distal tip, the first valve is closed prevent fluid in the bulb from flowing into the supply tube; the second valve is opened to allow fluid held in the bulb to flow from the bulb to the inflatable member to inflate the inflatable member; and the third valve is closed to prevent fluid from flowing from the inflatable member to the fluid reservoir; and in response to in response to a release of the compression of the bulb positioned in the distal tip: the first valve is opened to allow fluid to flow from the supply tube into the bulb to refill the bulb; the second valve is closed to prevent flow of fluid from the bulb into the supply tube; and the third valve is closed to prevent fluid from flowing from the inflatable member to the fluid reservoir.

In some implementations, in a second mode of operation of the implantable fluid operated inflatable device, in response to a depression of the button portion of the second fluid flow control device, the third valve is opened to allow fluid to flow from the inflatable member to the fluid reservoir in response to a force exerted on the third valve by the arm portion, to deflate the inflatable member; the first valve is closed prevent fluid in the bulb from flowing into the supply tube; and the second valve is closed to prevent fluid held in the bulb from flowing to the inflatable member.

In some implementations, the fluid flow control system, the inflatable member and the fluid reservoir are incorporated into a single unit.

### BRIEF DESCRIPTION OF THE DRAWINGS

FIG. 1 is a block diagram of an example implantable fluid operated inflatable device.

FIG. 2A is a perspective view of an example implantable fluid operated inflatable device, according to an aspect.

FIG. 2B is a cross-sectional view, taken along line A-A of FIG. 2A.

FIGS. 3A and 3B are cross-sectional views, taken along line A-A of FIG. 2A, illustrating a first mode of operation of the example implantable fluid operated inflatable device.

FIG. 3C is a cross-sectional view taken along line B-B of FIG. 2A, illustrating the flow a second mode of operation of the example implantable fluid operated inflatable device.

FIG. 4A illustrates an example distal tip portion of an example implantable fluid operated inflatable device.

FIG. 4B illustrates an example distal tip portion of an example implantable fluid operated inflatable device.

FIG. 5A is a cross-sectional view taken along line C-C of FIG. 2A, illustrating an example inflatable member 240 of an example implantable fluid operated inflatable device.

FIG. 5B is a cross-sectional view taken along line C-C of FIG. 2A, illustrating an example inflatable member 240 of an example implantable fluid operated inflatable device.

FIG. 5C is a cross-sectional view taken along line C-C of FIG. 2A, illustrating an example inflatable member 240 of an example implantable fluid operated inflatable device.

FIG. 6A illustrates features of an example actuation device of an example implantable fluid operated inflatable device.

FIG. 6B illustrates features of an example actuation device of an example implantable fluid operated inflatable device.

FIG. 6C illustrates features of an example actuation device of an example implantable fluid operated inflatable device.

FIG. 7A is a cross-sectional view of an example valve in an axial orientation.

FIG. 7B is a cross-sectional view of an example valve in a radial orientation.

### DETAILED DESCRIPTION

Detailed implementations are disclosed herein. However, it is understood that the disclosed implementations are merely examples, which may be embodied in various forms. Therefore, specific structural and functional details disclosed herein are not to be interpreted as limiting, but merely as a basis for the claims and as a representative basis for teaching one skilled in the art to variously employ the implementations in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having”, as used herein, are defined as comprising (i.e., open transition). The term “coupled” or “moveably coupled,” as used herein, is defined as connected, although not necessarily directly and mechanically.

In general, the implementations are directed to bodily implants. The term patient or user may hereinafter be used for a person who benefits from the medical device or the methods disclosed in the present disclosure. For example, the patient can be a person whose body is implanted with the medical device or the method disclosed for operating the medical device by the present disclosure.

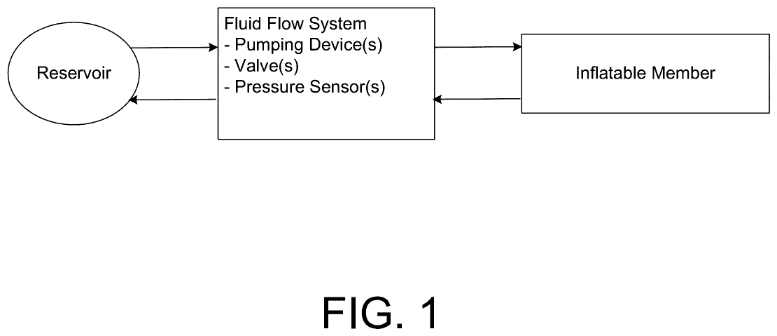

FIG. 1 is a block diagram of an example implantable fluid operated inflatable device. The example implantable fluid operated inflatable device may include an inflatable member that can be selectively inflated with fluid stored in a fluid reservoir. In a first mode of operation, for example, an inflation mode, or a pressurization mode, a fluid flow system may direct fluid from the fluid reservoir to the inflatable member, for inflation of the inflatable member. In a second mode of operation, for example, a deflation mode, or a depressurization mode, the fluid flow system may direct fluid from the inflatable member (in the inflated state) back to the fluid reservoir, for deflation of the inflatable member. In some examples, the fluid flow system may include one or more components provided in one or more fluid passageways connecting the fluid reservoir and the inflatable member. In some examples, the fluid flow system may include, for example, one or more pumping devices that cause fluid to flow from the fluid reservoir toward the inflatable member in the first mode of operation, and that cause fluid to flow from the inflatable member towards the fluid reservoir in the second mode of operation. In some examples, one or more valves may be provided in the fluid passageways to control the flow of fluid between the fluid reservoir and the inflatable member in the first mode of operation and in the second mode of operation. In some examples, one or more pressure sensors may be provided in the fluid passageways and/or at the fluid reservoir and/or at the inflatable member to detect and/or monitor a corresponding pressure in the implantable fluid operated inflatable device.

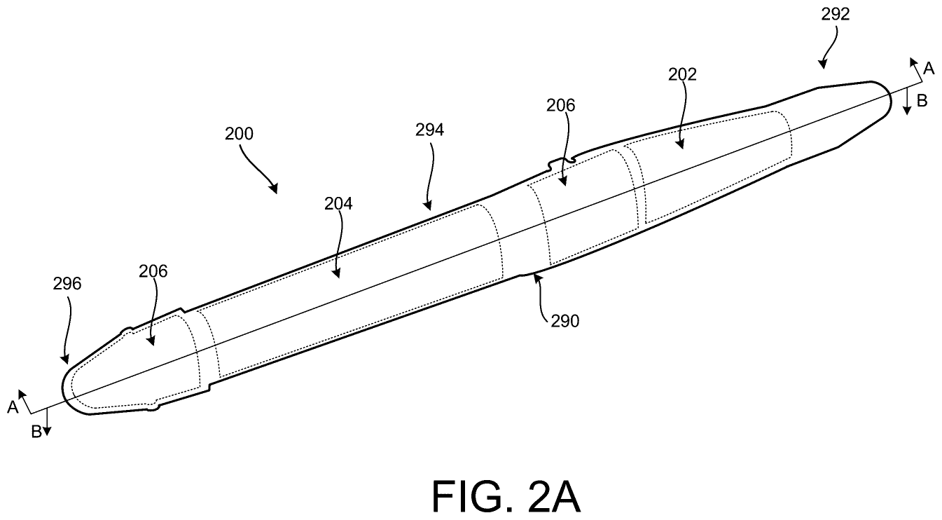

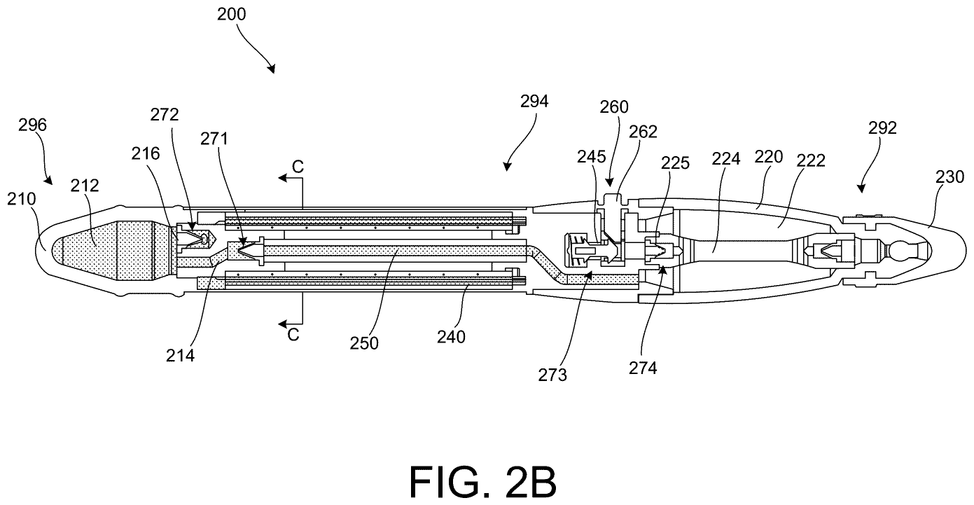

FIG. 2A is a perspective view of an example implantable fluid operated inflatable device 200, according to an aspect. FIG. 2B is a cross-sectional view of the example implantable fluid operated inflatable device 200, taken along line A-A of FIG. 2A. The example implantable fluid operated inflatable device 200 includes a reservoir portion 202 storing fluid for inflation of an inflatable portion 204. Fluid may flow from the reservoir portion 202 to the inflatable portion 204, via fluidics architecture including fluidics components received within the device 200, in response to manipulation of a fluid flow control system 206 for inflation of the inflatable portion 204. Fluid may flow from the inflatable portion 204, via the fluidics components received within the device 200, back to the reservoir portion 202, in response to manipulation of the fluid flow control system 206. In some examples, the fluid flow control system 206 may include one or more fluid flow devices providing for fluid flow in a first mode of operation, for example, an inflation mode, or a pressurization mode as described above. Manipulation of one or more of the fluid flow devices may actuate operation of the example device 200 in the first mode of operation, in which fluid flows from the reservoir portion 202 to the inflatable portion 204. In some examples, the fluid flow control system 206 may include one or more fluid flow devices providing for fluid flow in a second mode of operation, for example, a deflation mode, or a depressurization mode as described above. Manipulation of one or more of the fluid flow devices may actuate operation of the example device 200 in the second mode of operation, in which fluid flows form the inflatable portion 204 to the reservoir portion 202.

The reservoir portion 202, the inflatable portion 204, and the fluid flow control system 206 may be coupled in and/or on a housing 290 of the example device 200. In the example arrangement shown in FIG. 2A, the reservoir portion 202 is formed at a proximal end portion 292 of the example device 200, and the inflatable portion 204 is formed at an intermediate portion 294 of the example device 200. In the example arrangement shown in FIG. 2A, a first example fluid flow control device of the fluid flow control system 206 is provided at a distal end portion 296 of the example device 200, and a second example fluid flow control device of the fluid flow control system 206 is provided at the intermediate portion 294 of the example device 200. The first fluid flow control device and/or the second fluid flow control device may be manipulatable and/or controllable by the user to provide for fluid flow between the reservoir portion 202 and the inflatable portion 204 in accordance with a desired mode of operation of the example device 200.

The example implantable fluid operated inflatable device 200 shown in FIG. 2A is in the form of an inflatable penile prosthesis having all of the components of the example device 200 are incorporated into a single unit to provide for simplified implant of the device 200 in the patient, and simplified operation of the device 200 by the patient. The components of the example implantable fluid operated inflatable device 200 are incorporated into a single device, providing for simplified insertion in the patient, and simplified operation of the device 200 by the patient. The principles to be described herein may be applied to other types of implantable fluid operated inflatable devices, and in particular, to other types of implantable fluid operated inflatable devices that rely of the control of fluid flow to and from an inflatable portion of the device to achieve inflation, pressurization, activation, deflation, depressurization, deactivation, and the like. The example implantable fluid operated inflatable device 200 shown in FIG. 2A is a single piece implantable fluid operated inflatable device. The single piece implantable fluid operated inflatable device may provide for a simplified, less invasive, insertion into the patient than multi-piece devices including, for example, a separate fluid reservoir and/or separate fluid control device(s) including various pumps and/or valves and/or electronic controls. The single piece implantable fluid operated device may provide for simplified operation of the example device 200 by the user than multi-piece devices including, for example, a separate fluid reservoir and/or separate fluid control device(s) including various pumps and/or valves and/or electronic controls.

As shown in more detail in FIG. 2B, the example device 200 includes a distal tip 210 formed at the distal end portion 296, and a proximal tip 230 formed at the proximal end portion 292. In an example in which the device 200 is in the form of a penile prosthesis, the proximal tip 230 may facilitate the implanting and anchoring of the example device 200 in the body of the patient, and/or maintaining an implanted position and/or orientation of the example device 200 in the body of the patient. A fluid reservoir 220 is provided at the proximal end portion 292 of the example device 200, inward from and coupled to the proximal tip 230. Fluid is stored in an interior volume, or an interior space 222, defined within the fluid reservoir 220. In some examples, a stiffener 224 may be positioned in interior space 222 of the fluid reservoir 220. The stiffener 224 may extend between a proximal end portion and a distal end portion of the fluid reservoir 220. In some examples, the stiffener 224 may maintain an amount of rigidity in the reservoir portion 202 of the example device 200 in the implanted state, particularly in an inflated state of the inflatable portion 204 of the device 200, in which little to no fluid is held in the fluid reservoir 220.

An inflatable member 240 is installed in the intermediate portion 294 of the example device 200. The inflatable member 240 is in communication, for example, in fluid communication, with the fluid reservoir 220. In some examples, at least one fluid passageway provides for communication between the inflatable member 240 and the fluid reservoir 220. The at least one fluid passageway may allow fluid held in the fluid reservoir 220 to be selectively supplied to the inflatable member 240 for inflation of the inflatable member 240. The at least one fluid passageway may allow fluid filled in the inflatable member 240 to be returned to the fluid reservoir 220 for deflation of the inflatable member 240. In the example arrangement shown in FIG. 2B, a supply tube 250 extends between the fluid reservoir 220 and a bulb 212 positioned in the distal tip 210.

In some examples, the distal tip 210 and the bulb 212 are malleable, such that manipulation of the distal tip 210/bulb 212 may draw fluid from the at least one fluid passageway into the bulb 212, and/or may direct fluid held in the bulb 212 into the inflatable member 240. In some examples, the distal tip 210 and bulb 212 may define a first fluid flow control device of the fluid flow control system 206 of the example device 200. That is, manipulation of the first fluid flow control device defined by the distal tip 210 and bulb 212 may initiate operation of the example device 200 in the first mode, i.e., in the inflation mode. In some examples, fluid may be drawn from the fluid reservoir 220, through the supply tube 250, and into the bulb 212 in response to manipulation of the bulb 212, i.e., a squeezing and/or pressing of the bulb 212. Fluid may flow from the bulb 212 and into the inflatable member 240 in response to a release of the squeezing and/or pressing of the bulb 212, to fill the inflatable member 240 with fluid and inflate the inflatable member 240.

In the example arrangement shown in FIG. 2B, a first valve 271 is positioned between the bulb 212 and a corresponding end portion of the supply tube 250. In the example arrangement shown in FIG. 2B, the first valve 271 is positioned at an end of the supply tube 250 that joins an inlet port 214 through which fluid from the fluid reservoir 220 may be introduced into the bulb 212. In some examples, the first valve 271 is a non-return valve, or a one-way valve that is oriented so as to prevent the flow of fluid from the bulb 212 into the supply tube 250, for example, during operation in the first mode, i.e., the inflation mode, to prevent the backflow of fluid from the bulb 212 into the supply tube 250 during inflation of the inflatable member 240. In the example arrangement shown in FIG. 2B, a second valve 272 is positioned between the bulb 212 and the inflatable member 240. In the example arrangement shown in FIG. 2B, the second valve 272 is positioned at an outlet port 216 of the bulb 212 through which fluid from the bulb 212 may flow to the inflatable member 240. In some examples, the second valve 272 is a non-return valve, or a one-way valve that is oriented so as to prevent the flow of fluid from the inflatable member 240 into the bulb 212, for example, after inflation of the inflatable member 240 has been achieved, so that a desired inflation pressure may be maintained in the inflatable member 240.

In the example shown in FIG. 2B, a third valve 273 is positioned between the fluid reservoir 220 and the inflatable portion 204 of the example device 200, i.e., between the fluid reservoir 220 and the inflatable member 240. In some examples, the third valve 273 is a non-return valve, or a one-way valve. In the first mode of operation, i.e., during inflation of the inflatable member 240 and/or when fluid pressure is to be maintained in the inflatable member 240 after inflation is complete, the third valve 273 is in an unactuated state, as shown in FIG. 2B. In the unactuated state, the third valve 273 prevents the flow of fluid from the inflatable member 240 back into the fluid reservoir 220, to maintain inflation pressure in the inflatable member 240, and thus prevent deflation of the inflatable member 240.

In some examples, the device 200 may include an actuation device 260. The actuation device 260, when actuated by the user, may actuate the third valve 273, to initiate operation of the example device 200 in the second mode of operation, i.e., the deflation mode, in which fluid flows from the inflatable member 240 back to the fluid reservoir 220 to deflate the inflatable member 240. In some examples, the actuation device 260, alone or together with the third valve 273, may define a second fluid flow control device of the fluid flow control system 206 of the example device 200. User manipulation of the actuation device 260 may move the third valve 273 to an actuated position. In the actuated position of the third valve 273, a fluid passageway 245 between the inflatable member 240 and the fluid reservoir 220 is open. The opening of the fluid passageway 245 between the inflatable member 240 and the fluid reservoir 220 allows fluid to flow from the inflatable member 240 into the fluid reservoir 220, providing for deflation of the inflatable member 240.

In some examples, a fourth valve 274 may be positioned at a port 225 of the fluid reservoir 220, providing for fluid communication between the fluid reservoir 220 and the fluid passageway 245. In some examples, the fourth valve 274 is a non-return valve, or a one-way valve that is oriented so as to prevent the flow of fluid out of fluid reservoir 220 when the example device 200 is in an unactuated, or deflated state. That is, the fourth valve 274 may prevent unintentional inflation of the inflatable member 240 due to unintended flow of fluid from the fluid reservoir 220 and into the supply tube 250 via the port 225.

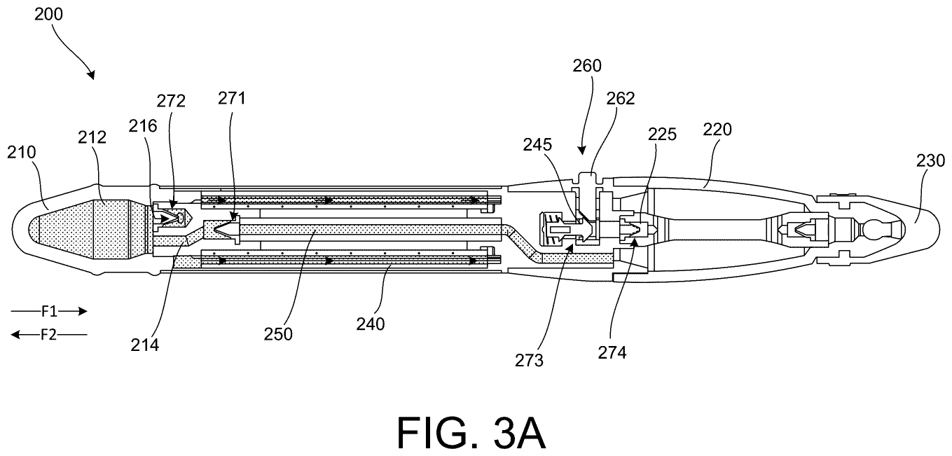

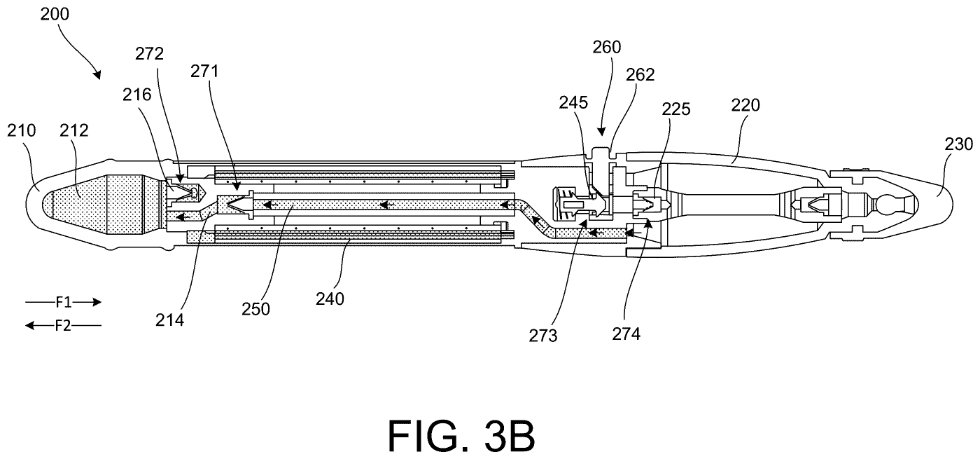

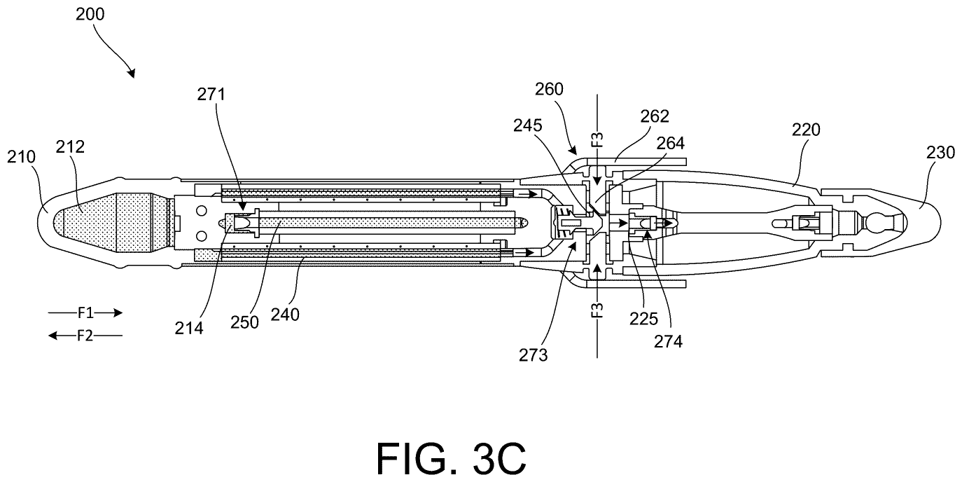

FIGS. 3A-3C are cross-sectional views of the example device 200, illustrating the flow of fluid through the example device 200. In particular, FIGS. 3A and 3B are cross-sectional views, taken along line A-A of FIG. 2A, illustrating the flow of fluid in the first mode of operation of the example device 200, i.e., during inflation of the inflatable member 240 of the example device 200. FIG. 3C is a cross-sectional view, taken along line B-B of FIG. 2A, illustrating the flow of fluid in the second mode of operation of the example device 200, i.e., during deflation of the inflatable member 240 of the example device 200.

Operation of the example device 200 in the first mode of operation and inflation of the inflatable member 240 will be described with reference to FIGS. 3A and 3B. To initiate operation of the example device 200 in the first mode of operation, and inflation of the inflatable member 240, the user may manipulate the distal tip 210/bulb 212. Manipulation of the distal tip 210/bulb 212 may include a pressing and/or squeezing of distal tip 210/bulb 212, for example, a repeated pressing/squeezing and releasing of the distal tip 210/bulb 212. An input at the bulb 212, in the form of a pressing and/or squeezing of the distal tip 210/bulb 212, may compress the bulb 212, as shown by the dotted lines in FIG. 3A. The compression of the bulb 212, and corresponding pressure generated, forces fluid held in the bulb 212 out through the outlet port 216 of the bulb 212. In particular, the pressure generated by the compression of the bulb 212 and corresponding flow of fluid out of the bulb 212 moves the second valve 272, positioned at the outlet port 216 of the bulb 212, in the direction of the arrow F1. This movement of the second valve 272 in the direction of the arrow F1 opens the outlet port 216, allowing the fluid held in the bulb 212 to flow to the inflatable member 240, to inflate the inflatable member 240. In the example arrangement shown in FIG. 3A, the pressure generated by the pressing/squeezing of the bulb 212 moves the first valve 271 in the direction of the arrow F2, thus closing off the inlet port 214, and restricting the flow of fluid between the bulb 212 and the supply tube 250, so that substantially all of the fluid held in the bulb 212 is available to flow to the inflatable member 240.

To continue operation of the example device 200 in the first mode of operation, and continue inflation of the inflatable member 240, the user may continue to manipulate the distal tip 210/bulb 212. For example, when the fluid held in the bulb 212 has been provided to the inflatable member 240 as described above with respect to FIG. 3A, the pressing/squeezing of the bulb 212 may be released, as shown in FIG. 3B. An input at the bulb 212, in the form of a release of the pressing/squeezing of the bulb 212 may generate a suction force in the bulb 212. This suction force generated at the bulb 212 may move the first valve 271 in the direction of the arrow F2, thus opening the inlet port 214 and allowing fluid to flow from the fluid reservoir 220, through the supply tube 250 and the inlet port 214, and into the bulb 212 to fill the bulb 212 with fluid. This suction force generated at the bulb 212 may draw the second valve 272 in the direction of the arrow F2, closing the outlet port 216, so that the fluid drawn into the bulb 212 remains in the bulb 212 as the bulb is refilled.

The pressing/squeezing of the bulb 212 as shown in FIG. 3A to convey fluid from the bulb 212 to the inflatable member 240, and then the release of the pressing/squeezing of the bulb 212 as shown in FIG. 3B to refill the bulb 212 with fluid, may be repeated until a desired level of inflation of the inflatable member 240 is achieved and/or a desired inflation pressure is achieved.

In the first mode of operation as described above with respect to FIGS. 3A and 3B, the first valve 271 and the second valve 272 may be alternately closed and opened to provide for the flow of fluid from the bulb 212 to the inflatable member 240, and from the fluid reservoir 220, through the supply tube 250 to the bulb 212 to refill the bulb 212 with fluid. While in the first mode of operation, i.e., the inflation mode, the third valve 273 may remain in a position and configuration that closes the fluid passageway 245 between the inflatable member 240 and the fluid reservoir 220, as shown in FIGS. 3A and 3B. That is, in the unactuated state shown in FIGS. 3A and 3B, the third valve 273 closes the fluid passageway 245, so as to maintain the fluid in the inflatable member 240 as the inflatable member 240 is inflated. Similarly, once the desired inflation pressure is achieved in the inflatable member 240, the third valve 273 may remain in a position that closes the fluid passageway 245, so that the fluid is maintained in the inflatable member 240 and the desired inflation pressure is maintained in the inflatable member 240. Once the desired inflation pressure has been achieved, the desired Inflation pressure in the inflatable member 240 is maintained with the first valve 271 positioned to close the inlet port 214, the second valve 272 positioned to close the outlet port 216, the third valve 273 positioned to close the fluid passageway 245, and the fourth valve 274 positioned to close the port 225 into the fluid reservoir 220.

Operation of the example device 200 in the second mode of operation and deflation of the inflatable member 240 will be described with reference to FIG. 3C. To initiate operation of the example device 200 in the second mode of operation, and depressurization or deflation of the inflatable member 240, the user may manipulate the actuation device 260 to actuate the third valve 273 as described above. In some examples, manipulation of the actuation device 260 includes a pressing of a button portion 262 of the actuation device 260, for example, in the direction of the arrow F3. In some examples, a configuration of the button portion 262 of the actuation device 260 is detectable at the outside of the example device 200, so that the button portion 262 can be located and manipulated by the user. An input at the actuation device 260, in the form of a manipulation of the button portion 262 of the actuation device 260, for example, a pressing and/or a pressing and holding of the button portion 262, may initiate operation of the example device 200 in the second mode, and depressurization and/or deflation of the inflatable member 240.

As shown in FIG. 3C, the actuation device 260 may include one or more arm portions 264 that extend radially inward from the button portion 262 of the actuation device 260. Inner peripheral end portions of the arm portion(s) 264 contact a corresponding surface of the third valve 273. In the example arrangement shown in FIG. 3C, a contour of the inner peripheral end portions of the arm portions 264 correspond to that of the contact surface of the third valve 273, such that movement of the arm portions 264 of the actuation device 260 causes reactionary movement of the third valve 273. In the example arrangement shown in FIG. 3C, the contour of the inner peripheral end portions of the arm portions 264 are angled, with the contact surface of the third valve 273 being oriented at a complementary angle. In this example arrangement, a pressing of the button portion 262 in the direction of the arrow F3 causes the arm portions 264 to move radially inward. This radially inward movement of the arm portions 264 causes the third valve 273 to move in the direction of the arrow F2, through the interaction between the inner peripheral end portions of the arm portions 264 and the corresponding outer contact surface of the third valve 273. Movement of the third valve 273 in the direction of the arrow F2 opens the fluid passageway 245, allowing fluid to flow from the inflatable member 240 back into the fluid reservoir 220 through the fluid passageway 245. The flow of fluid through the fluid passageway 245 causes the fourth valve 274 to move in the direction of the arrow F1, thus opening the port 225 into the fluid reservoir 220, and allowing the fluid to return to the fluid reservoir 220. Manipulation of the actuation device 260 in this manner, i.e., the pressing and holding of the button portion 262 of the actuation device 260, may be maintained until the desired level of depressurization or deflation is achieved. Once the desired level of depressurization and/or deflation has been achieved, the desired deflation pressure is maintained with the first valve 271 positioned to close the inlet port 214, the second valve 272 positioned to close the outlet port 216, the third valve 273 positioned to close the fluid passageway 245, and the fourth valve 274 positioned to close the port 225 into the fluid reservoir 220.

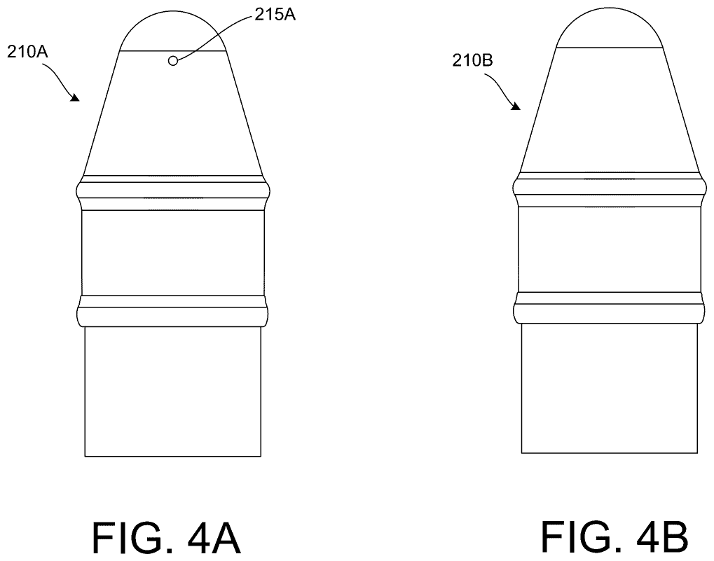

FIGS. 4A and 4B illustrate example configurations of the distal tip 210 which may be incorporated into the example implantable fluid operated inflatable device 200 described above with respect to FIGS. 2A-3C. In particular, FIG. 4A illustrates a first example distal tip 210A, and FIG. 4B illustrates a second example distal tip 210B. Either of the first example distal tip 210A or the second example distal tip 210B can be incorporated into the implantable fluid operated inflatable device 200 described above with respect to FIGS. 2A-3C. The first example distal tip 210A shown in FIG. 4A includes a needle guide opening 215A. In some examples, the needle guide opening 215A may provide for insertion of a needle and device-pulling suture through the first example distal tip 210A. The needle and device-pulling suture may facilitate and guide placement of the implantable fluid operated inflatable device 200 in the patient, so that the proximal tip 230 can be properly positioned and attached, and the first example distal tip 210A can be positioned in the distal corpus cavernosum. The second example distal tip 210B shown in FIG. 4B has a needless configuration.

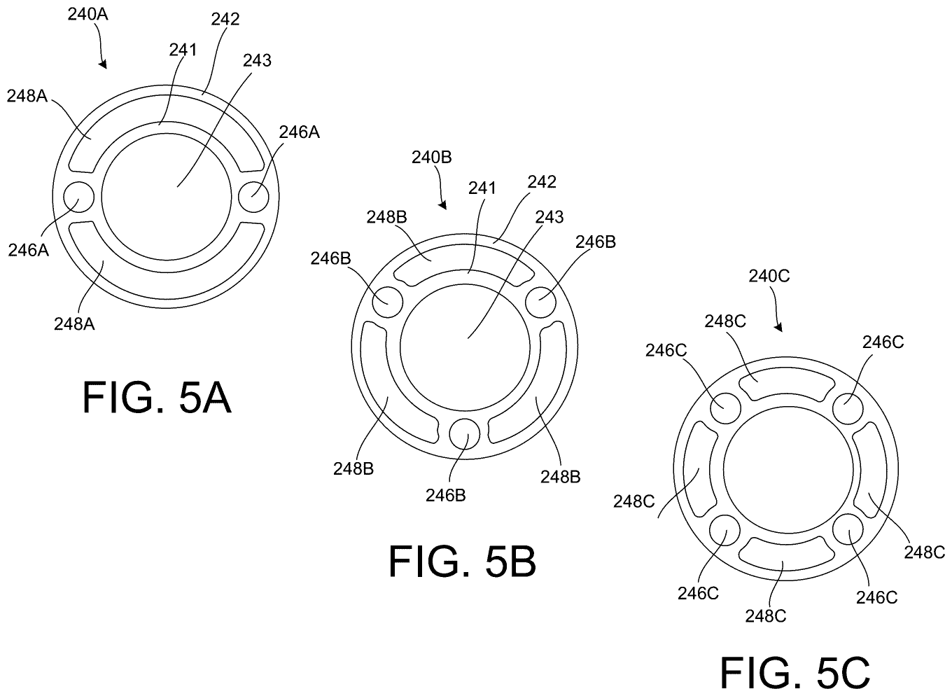

FIGS. 5A-5C are cross-sectional views taken along line C-C of FIG. 2A. FIGS. 5A-5C illustrate example configurations of the inflatable member 240 which may be incorporated into the example implantable fluid operated inflatable device 200 described above with respect to FIGS. 2A-3C. In particular, FIG. 5A is a cross-sectional view illustrating features of a first example inflatable member 240A, FIG. 5B is a cross-sectional view illustrating features of a second example inflatable member 240B, and FIG. 5C is a cross-sectional view illustrating features of a third example inflatable member 240C. Any one of the first example inflatable member 240A, the second example inflatable member 240B, or the third example inflatable member 240C can be incorporated into the implantable fluid operated inflatable device 200 described above with respect to FIGS. 2A-3C.

As shown in FIGS. 5A-5C, the first example inflatable member 240A, the second example inflatable member 240B, and the third example inflatable member 240C each include a first (inner) wall 241 and a second (outer) wall 242. In some examples, the first (inner) wall 241 and the second (outer) wall 242 may define an inflation tube that extends longitudinally along a corresponding portion of the example device 200. In some examples, the second (outer) wall 242 may define a corresponding portion of the housing 290. In each of the example arrangements shown in FIGS. 5A-5C, the first wall 241 and the second wall 242 each have a substantially circular cross-section, simply for purposes of discussion and illustration. In each of the example arrangements shown in FIGS. 5A-5C, the first wall 241 and the second wall 242 are substantially concentrically arranged, simply for purposes of discussion and illustration. In each of the example arrangements shown in FIGS. 5A-5C, a central opening 243 of the inflatable member 240 is defined by the first wall 241. In some examples, components of the implantable fluid operated inflatable device 200 may be received in the central opening 243. For example, as shown in FIGS. 2B-3C, the supply tube 250 may extend through the central opening 243 of the inflatable member 240. In some examples, a first valve body supporting the first valve 271 and/or the second valve 272 may at least partially extend into the central opening 243 at a distal end portion of the inflatable member 240, as shown in FIGS. 2B-3C. In some examples, a second valve body supporting the third valve 273 and/or the fourth valve 274 may at least partially extend into the central opening 243 at a proximal end portion of the inflatable member 240, as shown in FIGS. 2B-3C.

In some examples, the first wall 241 and/or the second wall 242 may be made of a fabric or woven material. In some examples, a silicon layer may be formed on an outer peripheral portion of the inflatable member 240. In some examples, the silicon layer on the outer peripheral portion of the inflatable member 240 may define a corresponding portion of the housing 290 of the example implantable fluid operated inflatable device 200.

As shown in FIG. 5A, the first example inflatable member 240A includes a first plurality of openings 246A and a second plurality of openings 248A defined between the first wall 241 and the second wall 242. In particular, the first example inflatable member 240A shown in FIG. 5A includes two substantially circular first openings 246A positioned between two substantially arcuate second openings 248A, thus defining two inflation sections of the first example inflatable member 240A. In some examples, the first plurality of openings 246A and/or the second plurality of openings 248A may be in communication with the outlet port 216 of the bulb 212, so that fluid from the bulb 212 can be filled in the first plurality of openings 246A and/or the second plurality of openings 248A to inflate the first example inflatable member 240A. In some examples, the first plurality of openings 246A and/or the second plurality of openings 248A may be in communication with the fluid passageway 245, so that fluid can flow from the first plurality of openings 246A and/or the second plurality of openings 248A to the fluid passageway 245 to deflate the first example inflatable member 240A.

In the example arrangement shown in FIG. 5A, the first plurality of openings 246A have a substantially circular cross-section, and the second plurality of openings 248A have a substantially arcuate cross-section, simply for purposes of discussion and illustration. The principles described herein can be applied to a configuration in which the first plurality of openings 246A and/or the second plurality of openings 248A have other cross-sectional shapes and/or contours and/or combinations thereof. In the example arrangement shown in FIG. 5A, the first plurality of openings 246A and the second plurality of openings 248A are alternately arranged about a central axis of the first example inflatable member 240A, simply for purposes of discussion and illustration. In the example arrangement shown in FIG. 5A, the first plurality of openings 246A and the second plurality of openings 248A are symmetrically arranged about a central plane of the first example inflatable member 240A, simply for purposes of discussion and illustration. The principles described herein can be applied to other arrangements of the first plurality of openings 246A and/or the second plurality of openings 248A.

As shown in FIG. 5B, the second example inflatable member 240B, a first plurality of openings 246B and a second plurality of openings 248B defined between the first wall 241 and the second wall 242. In particular, the second example inflatable member 240B shown in FIG. 5B includes three substantially circular first openings 246B respectively positioned between three substantially arcuate second openings 248B, thus defining three inflation sections of the second example inflatable member 240B. In some examples, the first plurality of openings 246B and/or the second plurality of openings 248B may be in communication with the outlet port 216 of the bulb 212, so that fluid from the bulb 212 can be filled in the first plurality of openings 246B and/or the second plurality of openings 248B to inflate the second example inflatable member 240B. In some examples, the first plurality of openings 246B and/or the second plurality of openings 248B may be in communication with the fluid passageway 245, so that fluid can flow from the first plurality of openings 246B and/or the second plurality of openings 248B to the fluid passageway 245 to deflate the second example inflatable member 240B.

In the example arrangement shown in FIG. 5B, the first plurality of openings 246B have a substantially circular cross-section, and the second plurality of openings 248B have a substantially arcuate cross-section, simply for purposes of discussion and illustration. The principles described herein can be applied to a configuration in which the first plurality of openings 246B and/or the second plurality of openings 248B have other cross-sectional shapes and/or contours and/or combinations thereof. In the example arrangement shown in FIG. 5B, the first plurality of openings 246B and the second plurality of openings 248B are alternately arranged about a central axis of the second example inflatable member 240B, simply for purposes of discussion and illustration. In the example arrangement shown in FIG. 5B, the first plurality of openings 246B and the second plurality of openings 248B are symmetrically arranged about a central plane of the second example inflatable member 240B, simply for purposes of discussion and illustration. The principles described herein can be applied to other arrangements of the first plurality of openings 246B and/or the second plurality of openings 248B.

As shown in FIG. 5C, the third example inflatable member 240C includes a first plurality of openings 246C and a second plurality of openings 248C defined between the first wall 241 and the second wall 242. In particular, the third example inflatable member 240C shown in FIG. 5C includes four substantially circular first openings 246C positioned between four substantially arcuate second openings 248C, thus defining four inflation sections of the third example inflatable member 240C. In some examples, the first plurality of openings 246C and/or the second plurality of openings 248C may be in communication with the outlet port 216 of the bulb 212, so that fluid from the bulb 212 can be filled in the first plurality of openings 246C and/or the second plurality of openings 248C to inflate the third example inflatable member 240C. In some examples, the first plurality of openings 246C and/or the second plurality of openings 248C may be in communication with the fluid passageway 245, so that fluid can flow from the first plurality of openings 246C and/or the second plurality of openings 248C to the fluid passageway 245 to deflate the third example inflatable member 240C.

In the example arrangement shown in FIG. 5C, the first plurality of openings 246C have a substantially circular cross-section, and the second plurality of openings 248C have a substantially arcuate cross-section, simply for purposes of discussion and illustration. The principles described herein can be applied to a configuration in which the first plurality of openings 246C and/or the second plurality of openings 248C have other cross-sectional shapes and/or contours and/or combinations thereof. In the example arrangement shown in FIG. 5C, the first plurality of openings 246C and the second plurality of openings 248C are alternately arranged about a central axis of the third example inflatable member 240C, simply for purposes of discussion and illustration. In the example arrangement shown in FIG. 5C, the first plurality of openings 246C and the second plurality of openings 248C are symmetrically arranged about a central plane of the third example inflatable member 240C, simply for purposes of discussion and illustration. The principles described herein can be applied to other arrangements of the first plurality of openings 246C and/or the second plurality of openings 248C.

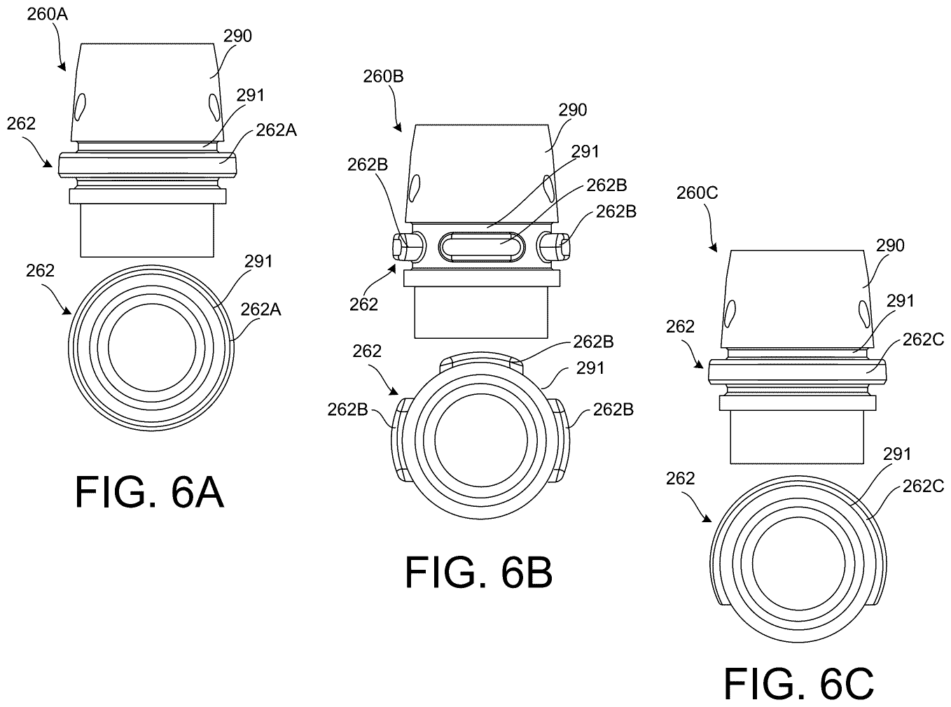

FIGS. 6A-6C each present a side view and an axial end view of example configurations of the actuation device 260 which may be incorporated into the example implantable fluid operated inflatable device 200 described above with respect to FIGS. 2A-3C. In particular, FIG. 6A illustrates features of a first example actuation device 260A, FIG. 6B illustrates features of a second example actuation device 260B, and FIG. 6C illustrates features of a third example actuation device 260C. Any one of the first example actuation device 260A, the second example actuation device 260B, or the third example actuation device 260C can be incorporated into the implantable fluid operated inflatable device 200 described above with respect to FIGS. 2A-3C.

As shown in FIGS. 6A-6C, first example actuation device 260A, the second example actuation device 260B, and the third example actuation device 260C each include the button portion 262 that is detectable and accessible to the user at an outer peripheral portion of the example implantable fluid operated inflatable device 200. In some examples, the button portion 262 may be disposed in a recessed portion 291 defined in an outer peripheral portion of the housing 290 of the example device 200, to accommodate and/or facilitate depression of the button portion 262 relative to the housing 290. As described above, depression of the button portion 262 of the actuation device 260 may move the one or more arm portion(s) 264 (not shown in FIGS. 6A-6C) radially inward, thus actuating the third valve 273 and opening the fluid passageway 245 between the inflatable member 240 and the fluid reservoir 220.

As shown in FIG. 6A, the button portion 262 of the first example actuation device 260A includes a single actuation button 262A positioned in the recessed portion 291 of the housing 290. In the example arrangement shown in FIG. 6A, the single actuation button 262A is a ring-shaped button that extends fully around the circumferential surface at the recessed portion 291 of the housing 290. In some examples, the single, ring-shaped actuation button 262A that extends fully around the housing 290 in this manner may increase a manipulation area of the button portion 262, thus facilitating user manipulation of the actuation device 260.

As shown in FIG. 6B, the button portion 262 of the second example actuation device 260B includes multiple actuation buttons 262B positioned in the recessed portion 291 of the housing 290. In the example arrangement shown in FIG. 6B, the multiple actuation buttons 262B are arranged along the circumferential surface at the recessed portion 291 of the housing 290, spaced apart from each other. This arrangement of the multiple actuation buttons 262B may generate a change in contour in the area of the actuation device 260 that improves the detectability of the actuation device 260 for the user, thus facilitating user manipulation of the actuation device 260. The example second example actuation device 260B shown in FIG. 6B includes three actuation buttons 262B, simply for purposes of discussion and illustration. More, or fewer, actuation buttons 262B may be provided in the recessed portion 291 of the housing 290, arranged similarly to, or differently from, the example arrangement shown in FIG. 6B.

As shown in FIG. 6C, the button portion 262 of the third example actuation device 260C includes a single actuation button 262A positioned in the recessed portion 291 of the housing 290. In the example arrangement shown in FIG. 6C, the single actuation button 262C extends partially around the circumferential surface at the recessed portion 291 of the housing 290. In some examples, the single actuation button 262B that extends partially around the housing 290 in this manner may improve the detectability of the actuation device 260 for the user, while also increasing a manipulation area of the button portion 262, thus facilitating user manipulation of the actuation device 260. The example third example actuation device 260C shown in FIG. 6C includes a single actuation button 262C that extends approximately two-thirds of the way around the outer circumferential surface at the recessed portion 291 of the housing 290 of the example device 200, simply for purposes of discussion and illustration. The single actuation button 262C can extend to cover a greater, or lesser, portion of the circumferential surface of the recessed portion 291 of the housing 290 than the example arrangement shown in FIG. 6C.

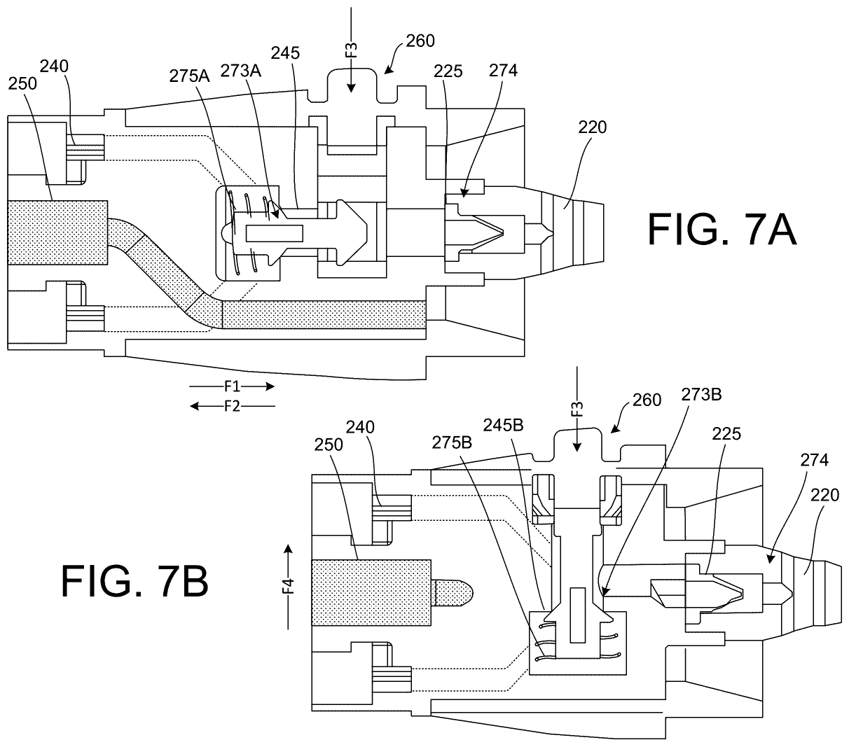

FIGS. 7A and 7B are close-in cross-sectional views illustrating interaction of the actuation device 260 with the third valve 273. In particular, FIG. 7A illustrates an axial orientation of an example third valve 273A in the example implantable fluid operated inflatable device 200. The axial orientation of the example third valve 273A shown in FIG. 7A is substantially the same as the example arrangement of the third valve 273 in the fluid passageway 245 as described above with respect to FIGS. 2B-3C, and thus duplicative detailed description will be omitted except where necessary for clarity. FIG. 7B illustrates a radial orientation of another example third valve 273B in the example implantable fluid operated inflatable device 200.

As shown in FIG. 7A, the example third valve 273A is positioned within the fluid passageway 245 connecting the inflatable member 240 and the fluid reservoir 220 (via the fourth valve 274 and the port 225 into the fluid reservoir 220). In the unactuated, or at rest, state, a biasing member 275A biases the example third valve 273A in the direction of the arrow F1, into a position to close the fluid passageway 245. As described above with respect to FIGS. 3A-3C, a position in which the example third valve 273A closes the fluid passageway 245 may block the flow of fluid from the inflatable member 240 to the fluid reservoir 220, thus maintaining a desired inflation pressure in the inflatable member 240 (including maintaining an inflated state of the inflatable member 240 or a deflated state of the inflatable member 240). A force applied to the button portion 262 of the actuation device 260 in the direction of the arrow F3 (to initiate operation in the second mode, or deflation of the inflatable member 240) may, in turn, exert a radial force on the example third valve 273A that moves the example third valve 273A in an axial direction, in the direction of the arrow F2, thus opening the fluid passageway 245 and allowing fluid to flow from the inflatable member 240 to the fluid reservoir 220.

In the example arrangement shown in FIG. 7B, the example third valve 273B is oriented radially, within a radially oriented fluid passageway 245B. In an unactuated, or at rest state, a biasing member 275B biases the example third valve 273B in the direction of the arrow F4, into a position to close the fluid passageway 245B. In this position, the example third valve 273B closes the fluid passageway 245B and blocks the flow of fluid from the inflatable member 240 to the fluid reservoir 220, thus maintaining a desired inflation pressure in the inflatable member 240 (including maintaining an inflated state of the inflatable member 240 or a deflated state of the inflatable member 240). A force applied to the button portion 262 of the actuation device 260 in the direction of the arrow F3 (to initiate operation in the second mode, or deflation of the inflatable member 240) may, in turn, exert a radial force on the example third valve 273B that moves the example third valve 273A in a radial direction, in the direction of the arrow F3, thus opening the fluid passageway 245B and allowing fluid to flow from the inflatable member 240 to the fluid reservoir 220.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the embodiments.

What is claimed is:

1. An implantable fluid operated inflatable device, comprising:
a housing;
a fluid reservoir received in the housing;
an inflatable member received in the housing;
fluidics components provided in the housing, the fluidics components providing for fluid communication between the fluid reservoir and the inflatable member; and
a fluid flow control system provided on the housing and configured to provide for fluid flow from the fluid reservoir to the inflatable member in a first mode of operation in response to an input at a first fluid flow control device of the fluid flow control system, and for fluid flow from the inflatable member to the fluid reservoir in a second mode of operation in response to an input at a second fluid flow control device of the fluid flow control system.

2. The implantable fluid operated inflatable device of claim 1, wherein the implantable fluid operated inflatable device is a penile prosthesis, including:
a proximal tip at a first end portion of the housing;
a distal tip at a second end portion of the housing; and
an inflatable portion corresponding to the inflatable member, at an intermediate portion of the housing.

3. The implantable fluid operated inflatable device of claim 2, wherein
the first fluid flow control device includes a malleable bulb provided in the distal tip; and
the second fluid flow control device includes a valve actuation device provided at the intermediate portion of the housing.

4. The implantable fluid operated inflatable device of claim 2, wherein the fluidics components include:
a supply tube connecting the fluid reservoir and a bulb positioned in the distal tip;
a first valve positioned in the supply tube;
a port connecting the bulb to the inflatable member;
a second valve positioned at the port connecting the bulb to the inflatable member;
a fluid passageway connecting the inflatable member and the fluid reservoir; and
a third valve positioned in the fluid passageway connecting the inflatable member and the fluid reservoir.

5. The implantable fluid operated inflatable device of claim 4, wherein the first fluid flow control device includes the bulb provided in the distal tip, and wherein, in response to a first manipulation of the bulb positioned in the distal tip:
the first valve is closed to prevent fluid in the bulb from flowing into the supply tube;
the second valve is opened to allow fluid held in the bulb to flow from the bulb to the inflatable member to inflate the inflatable member; and
the third valve is closed to prevent fluid from flowing from the inflatable member to the fluid reservoir.

6. The implantable fluid operated inflatable device of claim 5, wherein in response to a second manipulation of the bulb positioned in the distal tip:
the first valve is opened to allow fluid to flow from the supply tube into the bulb to refill the bulb;
the second valve is closed to prevent flow of fluid from the bulb into the supply tube; and
the third valve is closed to prevent fluid from flowing from the inflatable member to the fluid reservoir.

7. The implantable fluid operated inflatable device of claim 6, wherein the first manipulation of the bulb is a compression of the bulb and the second manipulation is a release of the compression of the bulb.

8. The implantable fluid operated inflatable device of claim 4, wherein the second fluid flow control device includes a valve actuation device provided at the intermediate portion of the housing, and wherein, in response to a manipulation of the valve actuation device:
the third valve is opened to allow fluid to flow from the inflatable member to the fluid reservoir to deflate the inflatable member;
the first valve is closed prevent fluid in the bulb from flowing into the supply tube; and
the second valve is closed to prevent fluid held in the bulb from flowing to the inflatable member.

9. The implantable fluid operated inflatable device of claim 8, wherein the valve actuation device includes:
a button portion provided in a recess formed in an outer peripheral surface of the housing; and
at least one arm extending radially inward, into the housing, from the button portion, so as to selectively contact the third valve.

10. The implantable fluid operated inflatable device of claim 9, wherein, in an unactuated state of the valve actuation device:
the third valve is positioned so as to close the fluid passageway between the inflatable member and the fluid reservoir.

11. The implantable fluid operated inflatable device of claim 9, wherein, in an actuated state of the valve actuation device:
the at least one arm is moved radially inward in response to a depression of the button portion;
the third valve moves to open the fluid passageway between the inflatable member and the fluid reservoir in response to a force exerted on the third valve by the at least one arm; and
fluid flows from the inflatable member to the fluid reservoir to deflate the inflatable member.

12. The implantable fluid operated inflatable device of claim 11, wherein the third valve is oriented axially within the housing, wherein the at least one arm is configured to exert a radial force on the third valve that moves the third valve in an axial direction to open the fluid passageway between the inflatable member and the fluid reservoir.

13. The implantable fluid operated inflatable device of claim 11, wherein the third valve is oriented radially within the housing, and wherein the at least one arm is configured to exert a radial force on the third valve that moves the third valve in a radial direction to open the fluid passageway between the inflatable member and the fluid reservoir.

14. The implantable fluid operated inflatable device of claim 9, wherein the button portion includes one of:
a ring-shaped button arranged in the recess, extending around the intermediate portion of the housing in which the recess is formed;
an arcuate button arranged in the recess, extending partially around the intermediate portion of the housing in which the recess is formed; or
a plurality of arcuate buttons arranged in the recess, spaced apart along the intermediate portion of the housing in which the recess is formed.

15. The implantable fluid operated inflatable device of claim 2, wherein the inflatable member includes:
an inner wall and an outer wall defining an inflation tube that extends longitudinally along the intermediate portion of the housing;
a plurality of first openings formed between the inner wall and the outer wall, extending longitudinally along a length of the inflatable member; and
a plurality of second openings formed between the inner wall and the outer wall, alternately arranged with the plurality of first openings, extending longitudinally along the length of the inflatable member,
wherein, in an inflated state of the inflatable member, the plurality of first openings and the plurality of second openings are configured to be filled with fluid so as to inflate the inflatable member.

16. The implantable fluid operated inflatable device of claim 1, wherein the inner wall defines a central opening extending longitudinally along a length of the inflatable member, wherein at least some of the fluidics components are received within a space defined by the central opening.

17. A fluid flow control system for an implantable fluid operated inflatable device, comprising:
a housing;
a first fluid flow control device provided at a distal end portion of the housing, the first fluid flow control device including:
a bulb provided in distal tip defining the distal end portion of the housing;
a first valve positioned in a supply tube connecting the bulb with a fluid reservoir; and
a second valve positioned in a port connecting the bulb and an inflatable member;
a second fluid flow control device provided at an intermediate portion of the housing, the second fluid flow control device including:
a valve actuation device configured to selectively actuate a third valve provided in a fluid passageway connecting the inflatable member and the fluid reservoir, the valve actuation device including:
a button portion provided in a recess formed in an outer peripheral surface of the housing; and
at least one arm extending radially inward, into the housing, from the button portion, so as to selectively contact the third valve.

18. The fluid flow control system of claim 17, wherein, in a first mode of operation of the implantable fluid operated inflatable device,
in response to a compression of the bulb positioned in the distal tip:
the first valve is closed prevent fluid in the bulb from flowing into the supply tube;
the second valve is opened to allow fluid held in the bulb to flow from the bulb to the inflatable member to inflate the inflatable member; and
the third valve is closed to prevent fluid from flowing from the inflatable member to the fluid reservoir; and
in response to in response to a release of the compression of the bulb positioned in the distal tip:
the first valve is opened to allow fluid to flow from the supply tube into the bulb to refill the bulb;
the second valve is closed to prevent flow of fluid from the bulb into the supply tube; and
the third valve is closed to prevent fluid from flowing from the inflatable member to the fluid reservoir.

19. The fluid flow control system of claim 17, wherein, in a second mode of operation of the implantable fluid operated inflatable device,
in response to a depression of the button portion of the second fluid flow control device:
the third valve is opened to allow fluid to flow from the inflatable member to the fluid reservoir in response to a force exerted on the third valve by the arm portion, to deflate the inflatable member;
the first valve is closed prevent fluid in the bulb from flowing into the supply tube; and
the second valve is closed to prevent fluid held in the bulb from flowing to the inflatable member.

20. The fluid flow control system of claim 17, wherein the fluid flow control system, the inflatable member and the fluid reservoir are incorporated into a single unit.
